(19) United States
(12) Patent Application Publication
(10) Pub. No.: US 2026/0196616 A1
(43) Pub. Date: Jul. 9, 2026

(54) ELECTROCHEMICAL CELLS FOR MEDICAL IMPLANTABLE DEVICE

(71) Applicant: Medtronic, Inc., Minneapolis, MN (US)

(72) Inventors: Christian S. Nielsen, River Falls, WI (US); Craig L. Schmidt, Eagan, MN (US)

(21) Appl. No.: 19/133,530

(22) PCT Filed: Nov. 28, 2023
(86) PCT No.: PCT/US2023/081263

§ 371 Date: May 28, 2025

Related U.S. Application Data
(60) Provisional application No. 63/428,148, filed on Nov. 28, 2022.

(51) Int. Cl. H01M 50/131; A61N 1/375; A61N 1/378; H01M 10/42; H01M 10/46; H01M 50/107

(52) U.S. Cl. CPC H01M 50/131; A61N 1/3756; A61N 1/3785; H01M 10/4257; H01M 10/46; H01M 50/107; H01M 2010/4271; H01M 2220/30

(57) ABSTRACT

Batteries having a passageway extending therethrough are described. A battery may extend along a longitudinal axis from a proximal end to a distal end. The battery may include a battery case, an anode disposed in the battery case, a cathode disposed in the battery case, and a passageway defined by the battery case. The battery case may define an exterior surface of the battery. The battery case may include an outer wall, an inner wall, a distal header coupled to the outer wall and the inner wall, and a proximal header coupled to the outer wall and the inner wall. The outer wall may define an outer perimeter of the battery case, the outer perimeter being orthogonal to the longitudinal axis. The passageway may extend through the battery from a proximal opening at the proximal end to a distal opening at the distal end.

### FIELD

The present disclosure relates to, among other things, batteries or electrochemical cells.

### TECHNICAL BACKGROUND

Batteries or electrochemical cells are generally used to provide power to devices when wired connections to external power sources may be undesirable or inconvenient. For example, batteries may be used to provide power to implantable medical devices implanted in a patient. In general, it may be desirable that such implantable medical devices be relatively small. However, as the size of implantable medical devices becomes smaller, the volume available to house a battery and electrical components also becomes smaller and more constricted. Accordingly, the geometry and size of the battery may contribute substantially to the overall size of implantable medical devices and electrical component placement therein as implantable medical devices become smaller.

### BRIEF SUMMARY

As described herein, smaller implantable medical devices with greater flexibility in electrical component placement and use can be achieved using batteries with passageways extending therethrough. Batteries as described herein may include a passageway extending from an opening in a proximal end to an opening in a distal end. The passageway may allow electrical components to be disposed in or move through the battery via the passageway. Furthermore, the passageway may allow for the wires or other electrical connections to be routed through the battery without or in addition to a feedthrough connection.

Described herein, among other things, is an implantable medical device extending along a longitudinal axis from a proximal device end to a distal device end. The implantable medical device may include a battery and a housing. The battery may extend along the longitudinal axis from a proximal battery end to a distal battery end. The battery may include a battery case defining an exterior surface of the battery, an anode disposed in the battery case, a cathode disposed in the battery case, an electrolyte disposed in the battery case, and a passageway extending through the battery from a proximal opening at the proximal battery end to a distal opening at the distal battery end. The passageway may be defined by the battery case. The housing may be coupled to the battery case. The housing and the battery case may cooperate to define an exterior surface of the implantable medical device.

In general, in one aspect, the present disclosure describes a battery extending along a longitudinal axis from a proximal end to a distal end. The battery may include a battery case, an anode disposed in the battery case, a cathode disposed in the battery case, and a passageway defined by the battery case. The battery case may define an exterior surface of the battery. The battery case may include an outer wall, an inner wall, a distal header coupled to the outer wall and the inner wall, and a proximal header coupled to the outer wall and the inner wall. One or both of the proximal header and the distal header are made up of any suitable conductive material (e.g., aluminum, copper, titanium, and the like). The outer wall may define an outer perimeter of the battery case. The outer perimeter may be orthogonal to the longitudinal axis. The passageway may extend through the battery from a proximal opening at the proximal end to a distal opening at the distal end.

Advantages and additional features of the subject matter of the present disclosure will be set forth in the detailed description which follows, and in part will be readily apparent to those skilled in the art from that description or recognized by practicing the subject matter of the present disclosure as described herein, including the detailed description which follows, the claims, as well as the appended drawings.

It is to be understood that both the foregoing general description and the following detailed description present embodiments of the subject matter of the present disclosure, and are intended to provide an overview or framework for understanding the nature and character of the subject matter of the present disclosure as it is claimed. The accompanying drawings are included to provide a further understanding of the subject matter of the present disclosure and are incorporated into and constitute a part of this specification. The drawings illustrate various embodiments of the subject matter of the present disclosure and together with the description serve to explain the principles and operations of the subject matter of the present disclosure. Additionally, the drawings and descriptions are meant to be merely illustrative and are not intended to limit the scope of the claims in any manner.

### BRIEF DESCRIPTION OF THE DRAWINGS

The following detailed description of specific embodiments of the present disclosure can be best understood when read in conjunction with the following drawings, in which:

FIG. 1 is a perspective view of an embodiment of an apparatus or device that includes a battery with a passageway;

FIG. 2 is a schematic perspective view of the device of FIG. 1;

FIG. 3 is a perspective view of an embodiment of the battery of the device of FIGS. 1 and 2;

FIG. 4 is an exploded view of the battery of FIG. 3.

FIG. 5 is a schematic view of the battery of FIGS. 3 and 4.

The schematic drawing is not necessarily to scale.

### DETAILED DESCRIPTION

Reference will now be made in greater detail to various embodiments of the subject matter of the present disclosure, one or more embodiments of which are illustrated in the accompanying drawings. Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same or similar to other numbered components.

As electronic devices become smaller, the volume available to house batteries and electrical components also becomes smaller and more constricted. To minimize device size, a portion of an exterior surface of a given device may be defined or formed by a battery of such device. However, when a battery of a device defines a portion of an exterior surface of the device, the arrangement of components within a housing of the device may become even more restricted. For example, if an outer diameter or perimeter of a device is defined by a battery, electrical components may be restricted to a single side of the battery, or one or more electrical feedthroughs may be needed to allow electrical connections between devices on opposite sides of the battery. Furthermore, movement of components designed to move within the housing of the device may be limited by the lack of any pathway to move around or through the battery within the housing.

Batteries that include a passageway that extends through the battery may allow for more flexibility in the arrangement of components within the device when the battery defines a portion of an exterior surface of the device. Batteries that include a passageway as described herein may allow electrical connections to be routed through the passageway, may allow components to be disposed in the passageway, or may allow components to move within or through the passageway. Furthermore, such batteries may allow the construction of smaller devices that retain at least some of the flexibility of component arrangement afforded by the space around batteries of larger devices.

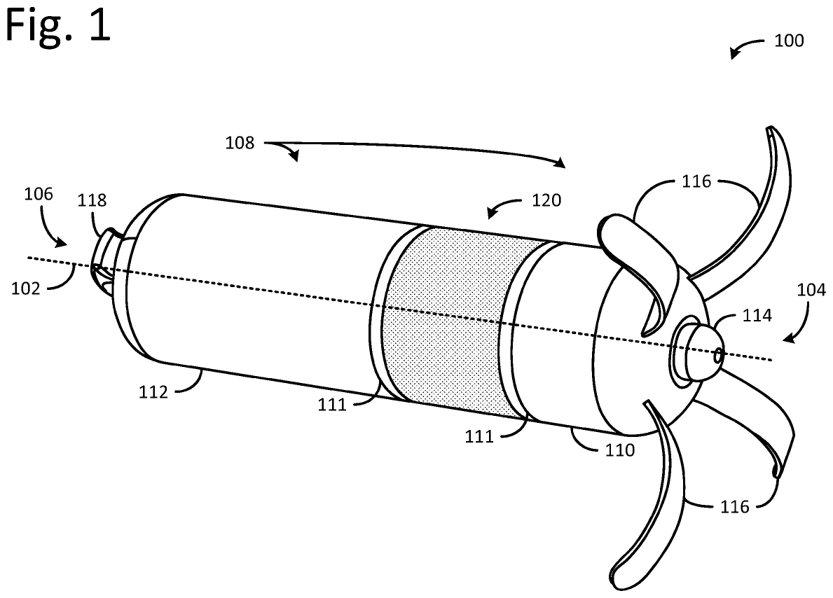

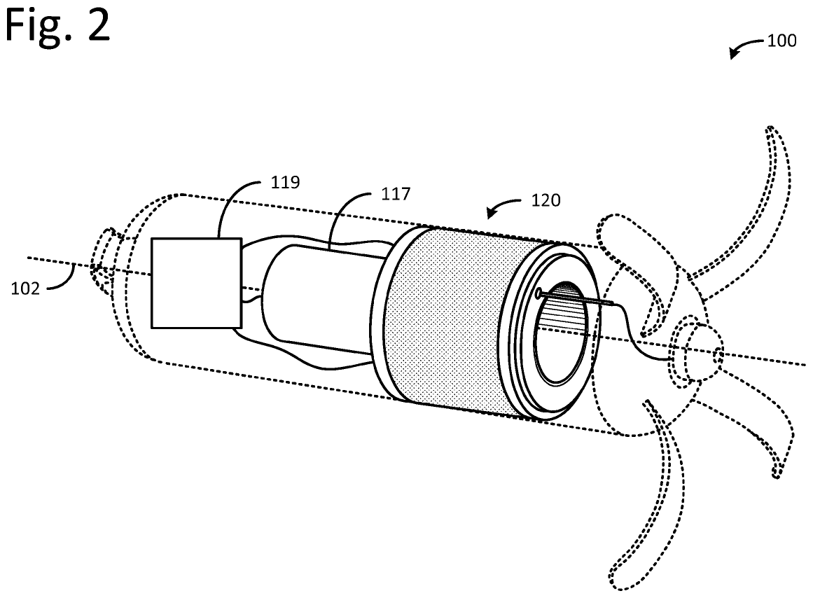

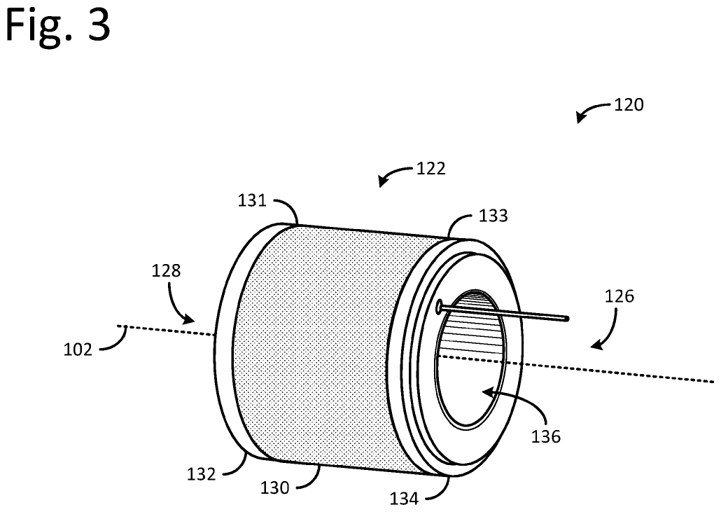

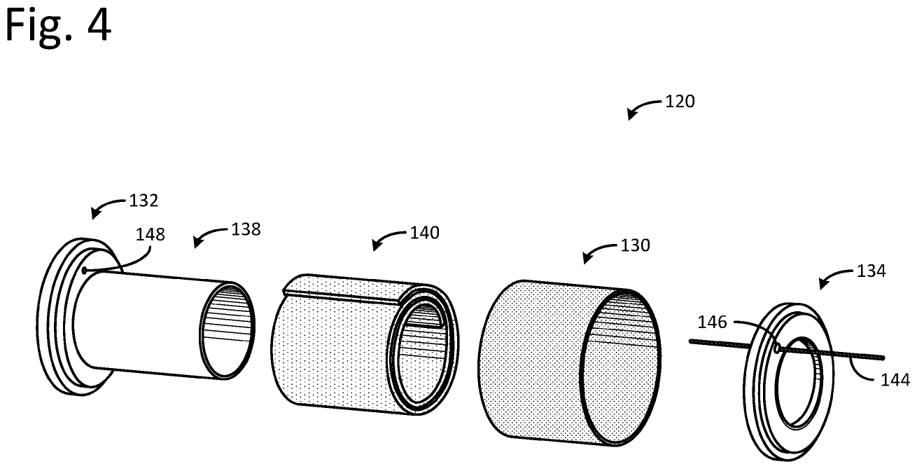

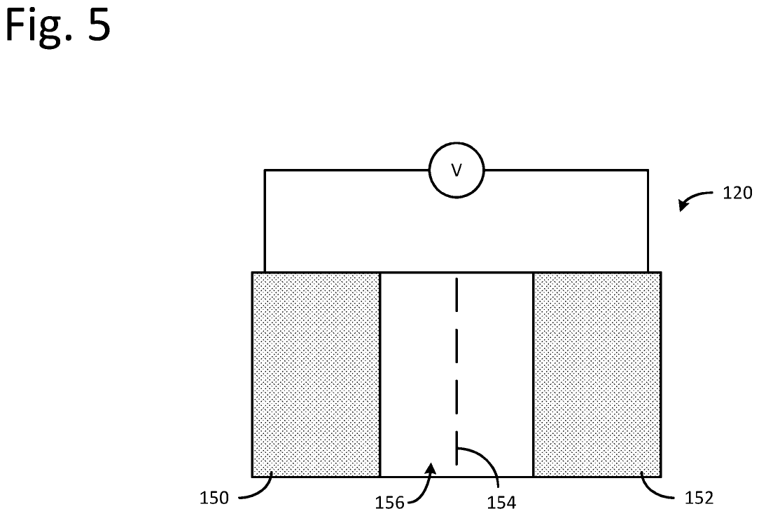

A device that includes a battery with a passageway as described herein, is depicted in FIGS. 1 and 2. FIG. 1 shows a perspective side view of a device 100 and FIG. 2 shows a schematic side view of the device 100 with interior components of the device 100 shown. Furthermore, additional details of a battery or electrochemical cell 120 of the device 100 of FIGS. 1 and 2 are depicted in FIGS. 3, 4, and 5. FIG. 3 shows a perspective view of the battery 120, FIG. 4 shows an exploded view of the battery 120, and FIG. 5 shows a schematic view of the battery 120.

The device 100 may extend along a longitudinal axis 102 from a proximal device end 104 to a distal device end 106. The device 100 may be any suitable electronic device or apparatus such as, for example, an implantable medical device, a sensor apparatus, a miniature camera, or other battery powered electronic device. In one or more embodiments, the device 100 may be a leadless pacing device. Regardless of the particular device or apparatus that device 100 may be, the device 100 may include at least the battery 120 and a housing 108.

The battery 120 may extend along the longitudinal axis 102 from a proximal battery end 126 to a distal battery end 128. The battery 120 may include a battery case 122, an anode 150, a cathode 152, an electrolyte 156, and a passageway 136. The battery case 122 may define an exterior surface of the battery 120. The battery case 122 may also define the passageway 136. Additionally, the battery case 122 may house the anode 150, the cathode 152, the electrolyte 156, and other battery components. The battery case 122 may define the general shape of the battery 120. For example, the battery case 122 may define a cylinder, a polyhedron, a frustum, a pyramid, a cone, or other 3-dimensional shape. In general, such shapes of the battery case 122 may be considered hollow due to the passageway 136 of the battery 120. In one or more embodiments, the battery case 122 defines a hollow cylinder that extends along the longitudinal axis 102.

The battery case 122 may include an outer wall 130, an inner wall 138, a distal header 132, and a proximal header 134. The outer wall 130 may define an outer perimeter or diameter of the battery case 122. The inner wall 138 may be disposed within the outer wall 130. Furthermore, the inner wall 138 may define at least a portion of the passageway 136. The distal header 132 may be coupled to the outer wall 130 and the inner wall 138. The distal header 132 may define the distal end 128 of the battery 120. The battery 120 may further include a hermetic seal 131 between the outer wall 130 and the distal header 132. The hermetic seal 131 may be formed by brazing, laser welding, etc. The proximal header 134 may be coupled to the outer wall 130 and the inner wall 138. The proximal header 134 may define the proximal end 126 of the battery 120. The battery 120 may further include a hermetic seal 133 between the outer wall 130 and the proximal header 134. The hermetic seal 133 may be formed by brazing, laser welding, etc. The joining of the proximal header 134 and the outer wall 130 and the distal header 132 and the outer wall is thermodynamically stable in the battery electrolyte 156.

Although shown with two headers, in one or more embodiments, the battery case 122 may only include a single header. For example, the battery case 122 may include only the distal header 132 and the outer wall 130 and the inner wall 138 may be formed monolithically or coupled together at the proximal end 126 of the battery 120.

The battery case 122 may include or be formed from any suitable material or materials such as, for example, aluminum, titanium, stainless steel, nickel, nickel coated ferrous steels, or other suitable materials. The battery case 122 may be electrically conductive, electrically insulative, or both. For example, the outer wall 130 may be electrically insulative while each of the distal header 132 and the proximal header 134 act as a current collector, e.g. a copper current collector, for one of the anode 150 or the cathode 152.

The anode 150 and the cathode 152 may be referred to collectively as electrodes 140. The electrodes 140 may be disposed in the battery case 122 between the outer wall 130 and the inner wall 138. The electrodes 140 may be wrapped or wound in a spiral or other configuration (e.g., an oval or other hollow shape) around the inner wall 138 such that the inner wall 138 acts as a mandrel for the electrodes 140. In a spiral configuration, the anode 150 or the cathode 152 may be wrapped around itself to form a plurality of layers or windings. However, the anode 150 or the cathode 152 may be wrapped to form a hollow shape that does not overlap itself and does not define a plurality of windings. Alternatively, one or both of the electrodes 140 may define a hollow shape that surrounds the inner wall 138. Accordingly, electrodes 140 that define such a hollow shape may be disposed or provided around the inner wall 138 without being wrapped around the inner wall 138.

The electrodes 140 may include any suitable conductive or active materials. The particular conductive or active materials may depend on the battery or electrochemical cell type of the battery 120. The battery 120 may be any suitable battery or electrochemical cell type such as, for example, nickel-cadmium, nickel-metal hydride, lithium metal, lithium ion, lithium metal, etc. In general, the anode 150 and the cathode 152 may be arranged such that they do not directly contact each other.

The battery 120 may also include a separator 154 arranged between the anode 150 and the cathode 152. In other words, the separator 154 may be provided intermediate to the anode 150 and the cathode 152. The separator 154 may be configured to prevent direct contact between the anode 150 and the cathode 152. The separator 154 may further be configured to allow transport of ionic charge carriers between the anode 150 and the cathode 152. The separator 154 may define a membrane forming a microporous layer. The separator 154 may include any suitable material or materials. The separator 154 may include, for example, one or more of a polymer, polyethylene, polypropylene, polyimide, cellulose, or other materials for forming a microporous layer.

The electrolyte 156 may transport positively charged ions between the anode 150 and the cathode 152. The electrolyte 156 may be disposed in the battery case 122 after the battery case 122 is assembled using a fill port 148. The electrolyte 156 may include any suitable material or materials such as, for example, lithium salts, lithium bis(trifluoromethylsulfonyl)imide (LiTFSI), lithium bis(pentafluoroethylsulfonyl) imide (LiBETI), lithium tris(trifluorosulfonyl) methide, lithium perchlorate (LiClO₄), lithium tetrafluoroborate (LiBF₄), lithium hexafluoroarsenate (LiAsF₆), lithium hexafluorophosphate (LiPF₆), or other solute capable of transporting ionic charge carriers. The electrolyte 156 may be a liquid electrolyte.

The passageway 136 may extend through the battery 120 from a proximal opening at the proximal battery end 126 to a distal opening at the distal battery end 128. The passageway 136 may be defined by the battery case 122. For example, each of the proximal and distal openings may be disposed in one of the headers 132, 134 and the width or diameter of the passageway 136 may be at least partially defined by the inner wall 138. The passageway 136 may extend along or parallel to the longitudinal axis 102. In one or more embodiments, the passageway 136 may be coaxial with the longitudinal axis 102. The passageway 136 may have any suitable width or diameter. Furthermore, the width or diameter of the passageway 136 may be uniform or variable along a length (as measured along the longitudinal axis 102) of the battery 120. In one or more embodiments, the width or diameter of the passageway 136 may be 3 millimeters or less. Additionally, the width or diameter of the passageway 136 may be at least 0.5 millimeters.

Although the passageway 136 may provide a path through the battery 120 for electrical connections or wires, the battery 120 may also include one or more feedthroughs 144. The one or more feedthroughs 144 may each provide an electrical connection through the battery 120. Such additional electrical connections through the battery 120 may be useful when the passageway 136 is occupied by or acts as a pathway for electrical, mechanical, or electromechanical components. The feedthroughs 144 may include one or more electrically conductive materials such as, for example, titanium, aluminum, copper, etc. The battery 120 may also include a feedthrough insulator 146. The feedthrough insulator 146 may be disposed in the battery case 122 or on the feedthrough 144 to insulate the feedthrough from the battery case 122, the electrodes 140, or other conductive components of the battery 120. The feedthrough insulator 146 may include one or more insulative materials such as, for example, glass, ceramic materials (e.g., alumina), or other suitable insulative materials.

The housing 108 and the battery case 122 may cooperate to define an exterior surface of the device 100. The housing 108 may be coupled to the battery case 122. In general, the housing 108 in combination with the battery case 122 may define the overall or general shape of the device 100. Furthermore, the housing 108 and the battery case 122 may provide an interior space or volume for additional components of the device 100 and protection from the environment for the additional components. For example, the housing and the battery case 122 may protect against dust or fluid ingress into the interior space of volume of the device. In one or more embodiments, the device 100 may include a hermetic seal 111 between the housing 108 and the battery case 122.

As shown, the housing includes a proximal portion 110 and a distal portion 112. The proximal portion 110 may extend along the longitudinal axis 102 from the battery case 122 towards the proximal device end 104. The distal portion 112 may extend along the longitudinal axis 102 from the battery case 122 towards the distal device end 106. In other words, the battery 120 may be arranged somewhere between the proximal device end 104 and the distal device end 106 but does not define either of the proximal device end 104 or the distal device end 106. However, in one or more embodiments, the battery 120 may define the proximal device end 104 or the distal device end 106 and the housing 108 may extend from the battery case 122 towards the other of the proximal device end 104 and the distal device end 106. A hermetic seal 111 may be disposed between each of portion 110, 112 of the housing 108 and the battery case 122. The hermetic seal 111 may be formed by brazing, laser welding, etc.

The housing 108 may include or be formed from any suitable material or materials such as, for example, titanium, titanium alloys, stainless steel, noble metal coated metallic enclosures (e.g., gold), etc. The housing 108 may be electrically conductive, electrically insulative, or both. For example, the proximal portion 110, or portions thereof, may be electrically conductive while the distal portion 112 may be electrically insulative.

In addition to the housing 108 and the battery 120, the device 100 may include one or more additional components. The device 100 may include mechanical structures or components such as, for example, one or more fixation elements 116 or a flange 118. Such mechanical structures may facilitate movement and placement of the device 100 in a target environment. The fixation elements 116 may be used or configured to retain a position of the device 100 in the target environment. The flange 118 may be used or configured to facilitate movement or manipulation the device 100 in the target environment. For example, the flange 118 may be attached to a guidewire or other tool to position an implantable medical device (e.g., the device 100) and the fixation elements 116 may retain a position of an implantable medical device once properly implanted in a patient.

The device 100 may also include various electrical components such as, for example, an electrode 114, an electromechanical device 117, control circuitry 119, or other suitable electrical components. The electrode 114 may facilitate sensing or electrical pulse delivery in a target environment. The electromechanical device 117 may include, for example, a kinetic energy harvester, an accelerometer, a gyroscope, motors, generators, or other electromechanical devices.

The control circuitry 119 may include any suitable hardware or devices to control various electrical or electromechanical devices that the device 100 may include. In general, the control circuitry 119 may be disposed in the housing 108 and may be operatively coupled to the battery 120. In one or more embodiments, the control circuitry 119 may include a power management system. The power management system may be disposed in the housing and may be operatively coupled to the battery 120. The power management system may be configured to control charging and discharging of the battery 120. For example, the control circuitry 119 may be operatively coupled to a kinetic energy harvester (e.g., electromechanical device 117). The kinetic energy harvester may be configured to move along the longitudinal axis including at least partially in or through the passageway 136 and produce electrical energy as the device 100 is moved. The control circuitry 119 may be configured to receive the electrical energy provided by the kinetic energy harvester and use such electrical energy to charge the battery 120.

In one or more embodiments, the control circuitry 119 may include a controller that includes one or more processors operatively coupled to the battery 120 and disposed in the housing 108. The controller may be configured to receive power from the battery 120 and deliver one or more therapeutic pulses. To deliver the one or more therapeutic pulses the controller may be operatively coupled to the electrode 114 and configured to deliver the one or more therapeutic pulses using the electrode 114.

The specific configuration of the control circuitry 119 may depend on a type of the device 100. The control circuitry 119 may include, e.g., one or more processors, logic gates, clocks, queues, Electro-Static Discharge (ESD) protection circuitry for input and output signals, line drivers and line decoders for interfacing to external devices, etc. The control circuitry 119 may be provided in a Field-Programmable Gate Array (FPGA), a circuit board, a system on a chip, a fixed or mobile computer system (e.g., a personal computer or minicomputer), implemented in software, etc.

The exact configuration of the control circuitry 119 is not limiting and essentially any device capable of providing suitable computing capabilities and signal processing capabilities (e.g., sensor data, control signals, battery management, etc.) may be used. Further, in one or more embodiments, data (e.g., sensor data, therapy data, etc.) may be analyzed by a user, used by another machine that provides output based thereon, etc. As described herein, a digital file may be any medium (e.g., volatile or non-volatile memory, a CD-ROM, a punch card, magnetic recordable tape, etc.) containing digital bits (e.g., encoded in binary, trinary, etc.) that may be readable and/or writeable by control circuitry 119 described herein. Also, as described herein, a file in user-readable format may be any representation of data (e.g., ASCII text, binary numbers, hexadecimal numbers, decimal numbers, audio, graphical) presentable on any medium (e.g., paper, a display, sound waves, etc.) readable and/or understandable by a user.

Devices (e.g., device 100) that include a battery (e.g., battery 120) with a passageway (e.g., passageway 136) as described herein, may provide additional space or volume for various components contained within the interior space or volume of such devices. Furthermore, the passageway of the battery may allow components to move through battery. Accordingly, components of such devices may not be restricted to a single side of the battery within the devices.

The invention is defined in the claims. However, below there is provided a non-exhaustive list of non-limiting examples. Any one or more of the features of these examples may be combined with any one or more features of another example, embodiment, or aspect described herein.

Example Ex1: An implantable medical device extending along a longitudinal axis from a proximal device end to a distal device end, the implantable medical device comprising: a battery extending along the longitudinal axis from a proximal battery end to a distal battery end, the battery comprising: a battery case defining an exterior surface of the battery; an anode disposed in the battery case; a cathode disposed in the battery case; an electrolyte disposed in the battery case; and a passageway extending through the battery from a proximal opening at the proximal battery end to a distal opening at the distal battery end, the passageway defined by the battery case; and a housing coupled to the battery case, the housing and the battery case cooperating to define an exterior surface of the implantable medical device.

Example Ex2: The device as in example Ex1, wherein the battery case defines a hollow cylinder extending along the longitudinal axis.

Example Ex3: The device as in example Ex1, wherein the passageway extends along or parallel to the longitudinal axis.

Example Ex4: The device as in any one of the previous examples, wherein the passageway is coaxial with the longitudinal axis.

Example Ex5: The device as in any one of the previous examples, further comprising a kinetic energy harvester at least partially disposed in the passageway.

Example Ex6: The device as in any one of the previous examples, wherein the battery further comprises a feedthrough extending through the battery housing and electrically insulated from the battery.

Example Ex7: The device as in any one of the previous examples, wherein the battery case comprises: an outer cylinder wall defining an exterior surface of the implantable medical device; an inner cylinder wall defining a diameter of the passageway; a distal header coupled to the outer cylinder wall and the inner cylinder wall, the distal header defining the distal battery end; and a proximal header coupled to the outer cylinder wall and the inner cylinder wall, the proximal header defining the proximal battery end.

Example Ex8: The device as in any one of the previous examples, wherein the electrolyte comprises a liquid electrolyte.

Example Ex9: The device as in any one of the previous examples, further comprising one or more electrodes disposed on the housing.

Example Ex10: The device as in any one of the previous examples, further comprising a power management system disposed in the housing and operatively coupled to the battery and configured to control charging and discharging of the battery.

Example Ex11: The device as in any one of the previous examples, further comprising a hermetic seal between the housing and the battery case.

Example Ex12: The device as in any one of the previous examples, further comprising one or more electrodes disposed on the housing.

Example Ex13: The device as in any one of the previous examples, further comprising a controller comprising one or more processors operatively coupled to the battery and disposed in the housing, the controller configured to: receive power from the battery; and deliver one or more therapeutic pulses.

Example Ex14: The device as in any one of the previous examples, the battery case comprises titanium.

Example Ex15: The device as in any one of the previous examples, wherein the housing comprises: a proximal portion extending along the longitudinal axis from the battery case towards the proximal device end; and a distal portion extending along the longitudinal axis from the battery case towards the distal device end.

Example Ex16: The device as in any one of the previous examples, wherein a diameter of the passageway is 3 millimeters or less.

Example Ex17: The device as in any one of the previous examples, wherein the implantable medical device is a leadless pacing device.

Example Ex18: A battery extending along a longitudinal axis from a proximal end to a distal end, the battery comprising: a battery case defining an exterior surface of the battery, the battery case comprising: an outer wall defining an outer perimeter of the battery case; an inner wall; a distal header coupled to the outer wall and the inner wall; and a proximal header coupled to the outer wall and the inner wall; an anode disposed in the battery case; a cathode disposed in the battery case; and a passageway extending through the battery from a proximal opening at the proximal battery end to a distal opening at the distal battery end, the passageway defined by the battery case.

Example Ex19: The battery as example Ex18, wherein the battery case defines a hollow cylinder extending along the longitudinal axis.

Example Ex20: The battery as in any one of examples Ex18 or Ex19, wherein the passageway extends along or parallel to the longitudinal axis.

Example Ex21: The battery as in any one of examples Ex18 to Ex20, wherein the passageway is coaxial with the longitudinal axis.

Example Ex22: The battery as in any one of examples Ex18 to Ex21, further comprising a kinetic energy harvester operatively coupled to the battery and at least partially disposed in the passageway.

Example Ex23: The battery as in any one of examples Ex18 to Ex22, wherein the battery further comprises a feedthrough extending through the battery case and electrically insulated from the battery.

Example Ex24: The battery as in any one of examples Ex18 to Ex23, wherein: the outer wall and the inner wall each define a hollow cylinder; the distal header defines the distal end; and the proximal header defines the proximal end.

Example Ex25: The battery as in any one of examples Ex18 to Ex24, further comprising a liquid electrolyte disposed in the battery case.

Example Ex26: The battery as in any one of examples Ex18 to Ex25, further comprising: a hermetic seal between the outer wall and the distal header; and another hermetic seal between the outer wall and the proximal header.

Example Ex27: The battery as in any one of examples Ex18 to Ex26, the battery case comprises titanium.

Example Ex28: The battery as in any one of examples Ex18 to Ex27, wherein a diameter of the passageway is 3 millimeters or less.

Example Ex29: An apparatus comprising: a battery extending along a longitudinal axis from a proximal battery end to a distal battery end, the battery comprising: a battery case defining an exterior surface of the battery; an anode disposed in the battery case; a cathode disposed in the battery case; an electrolyte disposed in the battery case; and a passageway extending through the battery from a proximal opening at the proximal battery end to a distal opening at the distal battery end, the passageway defined by the battery case; and a housing coupled to the battery case, the housing and the battery case cooperating to define an exterior surface of the apparatus.

Example Ex30: The apparatus as in example Ex29, wherein the battery case defines a hollow cylinder extending along the longitudinal axis.

Example Ex31: The apparatus as in any one of examples Ex29 or Ex30, wherein the passageway extends along or parallel to the longitudinal axis.

Example Ex32: The apparatus as in any one of examples Ex29 to Ex31, wherein the passageway is coaxial with the longitudinal axis.

Example Ex33: The apparatus as in any one of examples Ex29 to Ex32, further comprising a kinetic energy harvester at least partially disposed in the passageway.

Example Ex34: The apparatus as in any one of examples Ex29 to Ex33, wherein the battery further comprises a feedthrough extending through the battery housing and electrically insulated from the battery.

Example Ex35: The apparatus as in any one of examples Ex29 to Ex34, wherein the battery case comprises: an outer cylinder wall defining an exterior surface of the apparatus; an inner cylinder wall defining a diameter of the passageway; a distal header coupled to the outer cylinder wall and the inner cylinder wall, the distal header defining the distal battery end; and a proximal header coupled to the outer cylinder wall and the inner cylinder wall, the proximal header defining the proximal battery end.

Example Ex36: The apparatus as in any one of examples Ex29 to Ex35, wherein the electrolyte comprises a liquid electrolyte.

Example Ex37: The apparatus as in any one of examples Ex29 to Ex36, further comprising one or more electrodes disposed on the housing.

Example Ex38: The apparatus as in any one of examples Ex29 to Ex37, further comprising a power management system disposed in the housing and operatively coupled to the battery and configured to control charging and discharging of the battery.

Example Ex39: The apparatus as in any one of examples Ex29 to Ex38, further comprising a hermetic seal between the housing and the battery case.

Example Ex40: The apparatus as in any one of examples Ex29 to Ex39, further comprising one or more electrodes disposed on the housing.

Example Ex41: The apparatus as in any one of examples Ex29 to Ex40, further comprising a controller comprising one or more processors operatively coupled to the battery and disposed in the housing, the controller configured to: receive power from the battery; and deliver one or more therapeutic pulses.

Example Ex42: The apparatus as in any one of examples Ex29 to Ex41, the battery case comprises titanium.

Example Ex43: The apparatus as in any one of examples Ex29 to Ex42, wherein the housing comprises: a proximal portion extending along the longitudinal axis from the battery case towards the proximal apparatus end; and a distal portion extending along the longitudinal axis from the battery case towards the distal apparatus end.

Example Ex44: The apparatus as in any one of examples Ex29 to Ex43, wherein a diameter of the passageway is 3 millimeters or less.

Example Ex45: The apparatus as in any one of examples Ex29 to Ex44, wherein the apparatus is an implantable medical device.

All scientific and technical terms used herein have meanings commonly used in the art unless otherwise specified. The definitions provided herein are to facilitate understanding of certain terms used frequently herein and are not meant to limit the scope of the present disclosure.

As used herein, singular forms “a,” “an” and “the” include plural referents unless the context clearly dictates otherwise. As used in this specification and the appended claims, the term “or” is generally employed in its sense including “and/or” unless the content clearly dictates otherwise. The term “and/or” means one or all of the listed elements or a combination of any two or more of the listed elements.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

It will be apparent to those skilled in the art that various modifications and variations can be made to the present inventive technology without departing from the spirit and scope of the disclosure. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

1. A battery extending along a longitudinal axis from a proximal end to a distal end, the battery comprising:
a battery case defining an exterior surface of the battery, the battery case comprising:
an outer wall defining an outer perimeter of the battery case;
an inner wall;
a distal header coupled to the outer wall and the inner wall; and
a proximal header coupled to the outer wall and the inner wall;
an anode disposed in the battery case;
a cathode disposed in the battery case; and
a passageway extending through the battery from a proximal opening at the proximal battery end to a distal opening at the distal battery end, the passageway defined by the battery case.

2. The battery as in claim 1, wherein the battery case defines a hollow cylinder extending along the longitudinal axis.

3. The battery as in claim 1, wherein the passageway extends along or parallel to the longitudinal axis.

4. The battery as in claim 1, wherein the passageway is coaxial with the longitudinal axis.

5. The battery as in any one of claim 1, further comprising a kinetic energy harvester operatively coupled to the battery and at least partially disposed in the passageway.

6. The battery as in claim 1, wherein:
the outer wall and the inner wall each define a hollow cylinder;
the distal header defines the distal end; and
the proximal header defines the proximal end.

7. The battery as in claim 1, further comprising:
a hermetic seal between the outer wall and the distal header; and
another hermetic seal between the outer wall and the proximal header.

8. The battery as in claim 1, wherein a diameter of the passageway is 3 millimeters or less.

9. An implantable medical device extending along a longitudinal axis from a proximal device end to a distal device end, the implantable medical device comprising:
a battery extending along the longitudinal axis from a proximal battery end to a distal battery end, the battery comprising:
a battery case defining an exterior surface of the battery;
an anode disposed in the battery case;
a cathode disposed in the battery case;
an electrolyte disposed in the battery case; and
a passageway extending through the battery from a proximal opening at the proximal battery end to a distal opening at the distal battery end, the passageway defined by the battery case; and
a housing coupled to the battery case, the housing and the battery case cooperating to define an exterior surface of the implantable medical device.

10. The device as in claim 9, further comprising one or more electrodes disposed on the housing.

11. The device as in claim 9, further comprising a power management system disposed in the housing and operatively coupled to the battery and configured to control charging and discharging of the battery.

12. The device as in claim 9, further comprising a controller comprising one or more processors operatively coupled to the battery and disposed in the housing, the controller configured to:
receive power from the battery; and
deliver one or more therapeutic pulses.

13. The device as in claim 9, wherein the housing comprises:
a proximal portion extending along the longitudinal axis from the battery case towards the proximal device end; and
a distal portion extending along the longitudinal axis from the battery case towards the distal device end.

14. The device as in claim 9, wherein the implantable medical device is a leadless pacing device.

15. An apparatus comprising:
a battery extending along a longitudinal axis from a proximal battery end to a distal battery end, the battery comprising:
a battery case defining an exterior surface of the battery;
an anode disposed in the battery case;
a cathode disposed in the battery case;
an electrolyte disposed in the battery case;
a passageway extending through the battery from a proximal opening at the proximal battery end to a distal opening at the distal battery end, the passageway defined by the battery case; and
a housing coupled to the battery case, the housing and the battery case cooperating to define an exterior surface of the apparatus.

16. The apparatus as in claim 15, wherein the battery case defines a hollow cylinder extending along the longitudinal axis.

17. The apparatus as in claim 15, wherein the passageway extends along or parallel to the longitudinal axis.

18. The apparatus as in claim 15, wherein the passageway is coaxial with the longitudinal axis.

19. The apparatus as in claim 15, further comprising a kinetic energy harvester operatively coupled to the battery and at least partially disposed in the passageway.

20. The apparatus as in claim 15, wherein a diameter of the passageway is 3 millimeters or less.
